(19) United States
(12) Patent Application Publication
(10) Pub. No.: US 2022/0093086 A1
(43) Pub. Date: Mar. 24, 2022

(54) METHOD AND A SYSTEM FOR CAPTURING CONVERSATIONS

(71) Applicant: Prama, Inc., Louisville, KY (US)

(72) Inventor: Srinivasa Raju Eedarapalli, Louisville, KY (US)

(73) Assignee: Prama, Inc.

(21) Appl. No.: 17/479,317

(22) Filed: Sep. 20, 2021

Related U.S. Application Data
(60) Provisional application No. 63/080,317, filed on Sep. 18, 2020.

(51) Int. Cl. G10L 15/183; G10L 15/22; G10L 15/05; G06F 3/16

(52) U.S. Cl. CPC G10L 15/183; G10L 15/22; G10L 2015/223; G06F 3/167; G10L 15/05

(57) ABSTRACT

The invention relates to method and system for capturing a conversation between a plurality of users. The method includes receiving voice inputs from a first user and a second user; segregating the voice inputs into a plurality of voice-fragments; converting the plurality of voice-fragments into a plurality of text inputs; identifying a context of the conversation from the plurality of text inputs; classifying the voice-fragments into a first-user category and a second user category; fetching a plurality of user profiles from a database; mapping the context of the conversation with user data of the plurality of user profiles, and the first-user category voice-fragments with the voice samples of the plurality of user profiles; determining an identity of the user based on the mapping; and updating the user profile of the identified user based on the context of the conversation.

### TECHNICAL FIELD

Generally, the invention relates to interactive user interfaces. More specifically, the invention relates to a method and a system for capturing a conversation between a plurality of users.

### BACKGROUND

User interfaces are the access points for users to interact with their user devices, smart devices, smart screens or software applications, e.g. web based or mobile based applications. With huge and ever-increasing developments in the smart devices and software applications, there is also an increase in requirement for more developed, interactive, usable, better interface designs of the user interfaces for making the user interaction with devices and applications smooth. Also, the users wish to put in minimum efforts while accessing their devices or applications, using the user interfaces, and getting their tasks done easily, rather than indulging in complex designs and user inputs required while interacting with user interfaces. Users may want to save their time and energy while using their devices and applications. As such, the users may want their direct interactions with their devices to be minimized, so that they are able to multitask.

To implement easy and smart ways to control the user devices and software applications, the user interfaces may have different formats, such as a Graphical User Interface (GUI) with touch screen ability, gesture-based interfaces and Voice User interfaces (VUIs).

For example, in GUI with touch screen ability or other forms of user inputs like a keypad, a keyboard or a mouse, a user needs to be in direct contact with the user device or an application. Also, the user needs to be precise while inputting an attribute using such GUIs which require more focus and attention. Additionally, due to direct interaction and precision, such GUIs may require more time and interaction, the user may not be able to multitask, for e.g. working on another device or task or job, while controlling one user device. Such direct interaction with more focus and precision, using devices like a keypad, keyboard or a mouse may be fairly time consuming and cost of this time when it is of experts such as doctors may be very high.

Gesture-based interface formats may also incur errors while using, because of required precision and focus in performing a proper gesture, and in addition performing the gesture in an accurate distinguishable dimensional area. Additionally, due to focused, precise and direct interaction, the user may not be able to multitask even in Gesture based interfaces, for e.g. working on another device or task or job, while controlling one user device. Thus, such Gesture-based interfaces may also be erroneous and difficult to use.

VUI formats have found much more applicability and implementation in today's smart world with a lot of user devices and software applications adopting VUIs. Many mobile based software applications are voice controlled, for e.g. the applications may interact with the user via a user voice command. Certain functions of the user devices and applications may be controlled via user voice commands. However, there remains a wide scope of improvement in such VUIs. A smooth interaction using voice commands is lacking in present VUIs. For example, because of poor speech-to-text recognition, relying only on voice commands may lead to errors. Also, there exists a need in the VUIs to be flexible and adaptable to variety of users and devices.

Furthermore, present speech-to-text recognition applied by the voice controlled user interfaces may have poor recognition of spoken words, poor suggestions to spoken words, and limited words in dictionary. Thus, the present speech-to-text recognition systems need to be efficient to be applicable in today's wide-ranging and comprehensive applications.

Thus, there is a need to address the above problems in the current voice based user interfaces and related speech-to-text recognition systems.

### SUMMARY

In one embodiment, a method for capturing a conversation between a plurality of users is disclosed. The method may include receiving voice inputs from a first user and a second user. It should be noted that the voice inputs may be obtained using one or more microphones positioned in the vicinity of each of the first user and the second user. The voice inputs may include voice attributes. The method may further include segregating the voice inputs into a plurality of voice-fragments. Each of the plurality of voice-fragments may be associated with one of the first user and the second user. The method may further include converting the plurality of voice-fragments into a plurality of text inputs, using a voice-to-text conversion model The method may further include identifying a context of the conversation from the plurality of text inputs. The method may further include classifying the voice-fragments into a first-user category and a second user category based on the voice attributes and the context of the conversation. The first-user category may be associated with the voice-fragments received from the first user and the second-user category may be associated with the voice-fragments received from the second user. The method may further include fetching a plurality of user profiles from a database. Each of the plurality of user profiles may include at least one of: a user data and a voice sample of the user. The method may further include mapping the context of the conversation with user data of the plurality of user profiles, and the first-user category voice-fragments with the voice samples of the plurality of user profiles. The method may further include determining an identity of the user based on the mapping. The method may further include updating the user profile of the identified user based on the context of the conversation.

In another embodiment, a system for capturing a conversation between a plurality of users is disclosed. The system may include a processor and a memory communicatively coupled to the processor. The memory may store processor-executable instructions, which, on execution, may cause the processor to receive voice inputs from a first user and a second use. It should be noted that the voice inputs may be obtained using one or more microphones positioned in the vicinity of each of the first user and the second user. The voice inputs may include voice attributes. The processor-executable instructions, on execution, may further cause the processor to segregate the voice inputs into a plurality of voice-fragments. Each of the plurality of voice-fragments may be associated with one of the first user and the second user. The processor-executable instructions, on execution, may further cause the processor to convert the plurality of voice-fragments into a plurality of text inputs, using a voice-to-text conversion model. The processor-executable instructions, on execution, may further cause the processor to identify a context of the conversation from the plurality of text inputs. The processor-executable instructions, on execution, may further cause the processor to classify the voice-fragments into a first-user category and a second user category based on the voice attributes and the context of the conversation. The first-user category may be associated with the voice-fragments received from the first user and the second-user category may be associated with the voice-fragments received from the second user. The processor-executable instructions, on execution, may further cause the processor to fetch a plurality of user profiles from a database. Each of the plurality of user profiles comprises at least one of: a user data and a voice sample of the user. The processor-executable instructions, on execution, may further cause the processor to map the context of the conversation with user data of the plurality of user profiles, and the first-user category voice-fragments with the voice samples of the plurality of user profiles. The processor-executable instructions, on execution, may further cause the processor to determine an identity of the user based on the mapping. The processor-executable instructions, on execution, may further cause the processor to update the user profile of the identified user based on the context of the conversation.

In yet another embodiment, a non-transitory computer-readable medium storing computer-executable instruction for capturing a conversation between a plurality of users is disclosed. The stored instructions, when executed by a processor, may cause the processor to perform operations including receiving voice inputs from a first user and a second user. It should be noted that the voice inputs may be obtained using one or more microphones positioned in the vicinity of each of the first user and the second user. The voice inputs may include voice attributes. The operations may further include segregating the voice inputs into a plurality of voice-fragments. Each of the plurality of voice-fragments may be associated with one of the first user and the second user. The operations may further include converting the plurality of voice-fragments into a plurality of text inputs, using a voice-to-text conversion model. The operations may further include identifying a context of the conversation from the plurality of text inputs. The operations may further include classifying the voice-fragments into a first-user category and a second user category based on the voice attributes and the context of the conversation. The first-user category may be associated with the voice-fragments received from the first user and the second-user category may be associated with the voice-fragments received from the second user. The operations may further include fetching a plurality of user profiles from a database. Each of the plurality of user profiles may include at least one of: a user data and a voice sample of the user. The operations may further include mapping the context of the conversation with user data of the plurality of user profiles, and the first-user category voice-fragments with the voice samples of the plurality of user profiles. The operations may further include determining an identity of the user based on the mapping. The operations may further include updating the user profile of the identified user based on the context of the conversation.

It is to be understood that both the foregoing general description and the following detailed description are exemplary and explanatory only and are not restrictive of the invention, as claimed.

### BRIEF DESCRIPTION OF THE DRAWINGS

The present application can be best understood by reference to the following description taken in conjunction with the accompanying drawing figures, in which like parts may be referred to by like numerals.

FIG. 1 illustrates a block diagram of an exemplary system for capturing a conversation between a plurality of users, in accordance with some embodiments of the present disclosure.

FIG. 2 illustrates a functional block diagram of various modules within a conversation capturing device, in accordance with some embodiments of the present disclosure.

FIG. 3 illustrates a flowchart of a method for capturing a conversation between a plurality of users, in accordance with some embodiments of the present disclosure.

FIG. 4 illustrates a flowchart of a method for classifying voice-fragments into different categories, in accordance with some embodiments of the present disclosure.

FIG. 5 illustrates a flowchart of a method for populating one or more predefined fields within user profiles, in accordance with some embodiments of the present disclosure.

FIG. 6 illustrates an exemplary flowchart for updating the user profile of the identified user, in accordance with an embodiment of the disclosure.

FIG. 7 illustrates an exemplary flowchart showing a method implementing a system of capturing conversation between the users, in accordance with some embodiments of the present disclosure.

FIGS. 8-12 illustrate exemplary graphical user interfaces (GUIs) with a voice command interface, implementing a conversation capturing device, showing an example of a user interacting with the graphical user interfaces through voice inputs, in accordance with an embodiment of the disclosure.

### DETAILED DESCRIPTION OF THE DRAWINGS

The following description is presented to enable a person of ordinary skill in the art to make and use the invention and is provided in the context of particular applications and their requirements. Various modifications to the embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention might be practiced without the use of these specific details. In other instances, well-known structures and devices are shown in block diagram form in order not to obscure the description of the invention with unnecessary detail. Thus, the present invention is not intended to be limited to the embodiments shown, but is to be accorded the widest scope consistent with the principles and features disclosed herein.

While the invention is described in terms of particular examples and illustrative figures, those of ordinary skill in the art will recognize that the invention is not limited to the examples or figures described. Those skilled in the art will recognize that the operations of the various embodiments may be implemented using hardware, software, firmware, or combinations thereof, as appropriate. For example, some processes can be carried out using processors or other digital circuitry under the control of software, firmware, or hard-wired logic (The term “logic” herein refers to fixed hardware, programmable logic and/or an appropriate combination thereof, as would be recognized by one skilled in the art to carry out the recited functions.) Software and firmware can be stored on computer-readable storage media. Some other processes can be implemented using analog circuitry, as is well known to one of ordinary skill in the art. Additionally, memory or other storage, as well as communication components, may be employed in embodiments of the invention.

The present disclosure provides a system and a related method, and a device implementing the system and the method for capturing a conversation between a plurality of users, by capturing user inputs using one or more user interfaces, such as a voice-enabled user interface. The recognition system captures users' inputs and recognizes commands from the user inputs in order to perform one or more functions. The system comprises a user interface, such as a voice enabled user interface, that captures user inputs; one or more modules to identify and recognize commands from the user inputs; one or more modules to process the user inputs, one or more modules to comprehend and understand the user inputs using one or more input recognition algorithms, one or more modules to automatically predict, correct and/or suggest a command and/or a context/text from the user inputs based on the comprehension; one or more modules to continuously learn, in real time, from the user manual inputs in situations of erroneous recognition by the system; and one or more modules to continuously update the system based on the real time learning.

The system may further include one or more databases, including a user database and a word database. The user database may store profiles of one or more users. A user profile may include and is not limited to user names, residing location, birth location, occupations, languages known to user, a preferred language, dialect, tones, accents, and the like of a user. It may be apparent to a person ordinary skilled in the art that the user database may include user related information (i.e., user data). The user related information, in some embodiments, may be required by the system to more appropriately understand the language and dialect of the user, without deviating from the meaning and scope of the present disclosure.

The word database may include one or more word dictionaries, one or more user-specific dictionaries based on user's language, dialect, tones, accents, pronunciation and the like.

In an embodiment, the user database and the word database may also reside on a network, such as a cloud network, with which the system may communicate and fetch information whenever required.

The system may further include one or more modules to listen through single or multi-users dialogues and conversations to comprehend the dialogues and conversations for automatically suggesting future context/text or answers from within the dialogues and conversations, thereby reducing users' need to manually input the text or answers.

The system may further comprise a weightage module that may provide a weightage or a rank related to the users of system. In an embodiment, the weightage module may provide ranks to the multiple users participating in a conversation. In an embodiment, the weightage module may provide a weightage to answers, conversations or inputs of each user when the system automatically listens or picks-up multiple users' inputs, conversations, or may also provide such weightage to user inputs, such as answers when the user is directly using the voice-enabled user interface. In an embodiment, the weightage module may also provide a weightage or a rank to one or more users, in real time, participating in a conversation, by gauging and comprehending dialogues of each user and ranking the users based on the level of importance of dialogues of each user in the conversation. Such weightage module is important in multi-user conversations, for the system to identify and pick the most probable and accurate answers based on the trust weightages to the answers or conversations and/or users, individually or in combination.

The weightage module provides weightage based on one or more weightage factors, including and not limited to a type of user, such as a primary user, a secondary user, a regular and frequent user, trust-based factor, occupation, and the like. It may be apparent to a person ordinary skill in the art that the weightage module may consider more factors while assigning weightage or ranks to the user, such as an input from the user to rank himself/herself, profession of the user, and the like, without deviating from the meaning and scope of the present disclosure.

For example, if a multi-user conversation involves a doctor and a patient, the weightage module of the recognition system may provide more trust weightage to the doctor than the patient. And, the system of the present disclosure may rank the dialogues of the doctor above than that of the patient to identify and suggest the probable and accurate answers. It may also be the situation, based on the real time conversation/dialogue analysis by the system, that the weightage module may provide more weightage to the patient's dialogues, in order to most accurately answer a future question.

Therefore, it may also be an ability of the system to provide real time weightage to the users, and further based on a particular question or situation in a conversation.

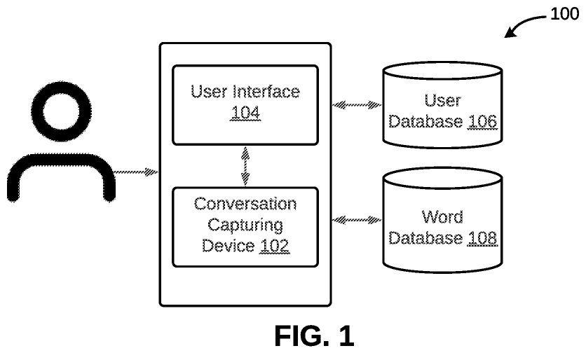

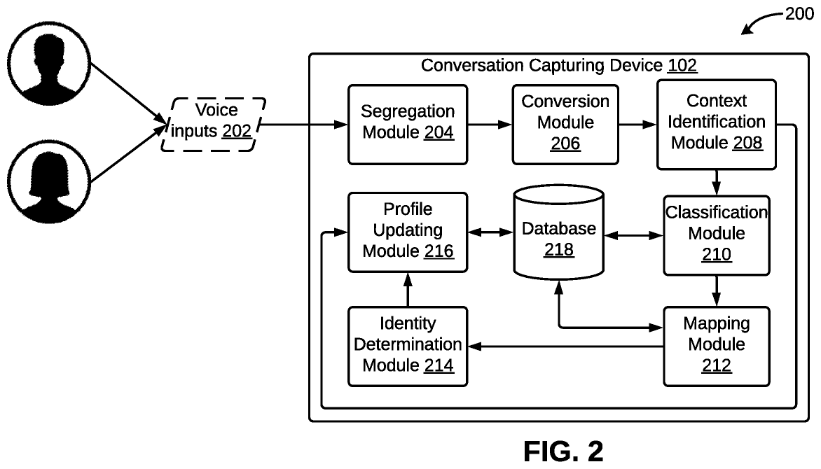

Referring to FIG. 1, a block diagram of an exemplary system 100 for capturing a conversation between a plurality of users is illustrated, in accordance with some embodiments of the present disclosure. The system 100 may include a conversation capturing device 102. The system 100 and associated conversation capturing device 102 may be deployed in a web-based environment or a device-based environment, such as a mobile device or a desktop. The system 100 may include a user interface 104, which is in communication with the conversation capturing device 102. In some embodiments, the user interface 104 may be within the conversation capturing device 102 along with various module, such as a recognition module for processing and recognizing user's inputs to further identify user commands from the captured inputs; a text generator for converting voice inputs into texts based on recognized user commands; a suggestion module for predicting, suggesting and/or correcting a command, text or context to convert the voice inputs; a weightage module for providing weightage or ranks related to one or more users of the system 100; and a database to store data (not shown in FIG. 1). The modules of the conversation capturing device 102 may be further explained in conjunction with FIG. 2.

The system 100 may include a user database 106 and a word database 108. In an embodiment, the word database 108 may include dictionaries and words specific to user profile, such as his/her profession, language, dialect, accent, and the like. In another embodiment, the word database 108 may include a user specific word database in addition to a generic word database 108. In an embodiment, the user specific word database keeps on updating based on real time learning.

In an embodiment, the user input interfaces or devices 104 may allow a user to interact with the conversation capturing device 102 and input any data content in the conversation capturing device 102. In an embodiment, the conversation capturing device 102 may also include one or more user output interfaces or devices (not shown in FIG. 1) allowing the conversation capturing device 102 to output any content to the user. The user input interface/device 104 may include and is not limited to voice enabled user interface, a touch-sensitive screen, non-touch sensitive screen, a touch keypad or a non-touch keypad, a keyboard, a mouse, a stylus, an image/video capturing device, such as camera, one or more push buttons, soft keys and the like. The output device may include and is not limited to display screen, a speaker, and the like.

The recognition module of the conversation capturing device 102 may identify and process the user inputs, such as a voice input. In an embodiment, the recognition module may include and is not limited to a voice recognition module, a text recognition module, an image recognition module, a video recognition module, and the like, based on the type of user input. In an example, when the user input is voice input using the voice enabled user interface, the voice recognition module may process the voice inputs and recognize language and dialect of the user to further identify user commands from the voice inputs.

In an embodiment, a user of the system 100 may provide or may interact with the conversation capturing device 102 via the user interface 104 (i.e, a voice-enabled user interface) to provide voice inputs to the conversation capturing device 102. In another embodiment, the conversation capturing device 102 may automatically listen or pick-up real-time monologues or dialogues of one or more users, in a single user or multi-user conversation environment. In another embodiment, the user is able to interact with the user input interface 104 to interact with the conversation capturing device 102 and input data content into the conversation capturing device 102, which is further captured by the conversation capturing device 102. Thus, for e.g. a user may interact with the touch sensitive screen to provide a text input to the conversation capturing device 102. Also, for an example, a user may interact with the camera to provide an image input to the conversation capturing device 102, which may be processed by the conversation capturing device 102.

After capturing the user inputs via the user interface 104, the conversation capturing device 102 may execute one or more recognition modules to process the user inputs for recognizing user's inputs, such as user's voice and user commands in the user inputs to perform one or more functions. The recognition module may implement one or more intent recognition algorithms for recognizing context and intent in the user commands from the user inputs, including voice input and other type of user inputs.

The user commands and the related one or more functions that may be recognized and performed by the conversation capturing device 102 may include and is not limited to recognizing a user control command from the user inputs to control a function attribute of a device or an application which implements the conversation capturing device 102; recognizing a selection command from the user input to select an attribute in a device or an application which implements the conversation capturing device 102; recognizing a text command from the user input, such as voice input to identify a voice input to be converted into text; recognizing a navigating command from the user input to navigate over one graphical UI of an application or between multiple GUIs of an application. It may be apparent to a person skilled in the art that the conversation capturing device 102 may perform more than one functions, apart from the aforementioned, based on the captured user inputs and processing of the user inputs, without deviating from the meaning and scope of the present disclosure.

Based on a recognized user command from the user inputs, the recognition module may process and execute that command on the web-based environment or the device-based environment, such as a mobile device or a desktop, into which the conversation capturing device 102 is deployed.

The device-based environment may include and is not limited to a smart mobile phone, a laptop, a desktop, an I-Pad, a tablet, a speaker, a display device, any other audio/video device with a microphone, and/or a software application downloaded and installed in the device that is controlled by the conversation capturing device 102, and the like, individually or in combination.

The web-based environment may include and is not limited to web-pages, web-based online forms, Web Based Questionnaires (Presented as Web Form, Survey or a Bot), and the like individually or in combination.

In an exemplary situation, if a user voice input is “increase the volume”, at a smart phone, the conversation capturing device 102 recognizes a function control command from the captured voice input, and processes and executes the command at the smart phone by increasing the volume.

In an exemplary situation, if a user wishes to enter a text using his voice input, for e.g. in a tab in an online form, the conversation capturing device 102 recognizes the text command from the captured voice input, and processes and executes the command and converts the voice input into text, using the text generator, and fills the tab in the online form.

In another exemplary situation, while filling online form at a web-page, the user wishes to navigate to a next question or tab to be filled or selected, the user may provide a voice input for e.g. “next question” or “next tab”, etc., the conversation capturing device 102 recognizes the navigation command, and processes and executes the command, and navigates to the next question or tab. Similarly, the user may provide a voice input for e.g. “next page”, etc., the conversation capturing device 102 recognizes the navigation command, and processes and executes the command, and navigates to the next page of an online form.

In an exemplary situation, a user interface of an online form in a web-based environment, presents a set of questions to answer at a given time. A user may answer the questions on this current set and move to the next set of questions, or go back to the previous set of questions, by providing the voice inputs for e.g. “next page”, “next question” or “next tab” or “previous question”, “skip question”, “skip page”, “delete answer and skip to next question”, and the like commands.

Thus, the voice commands may be speech-to-text based and use the text generator to convert speech into text and also may navigate between the question sets/pages, questions in a web-based environment or a device-based environment. The text generator may implement conventional speech-to-text algorithms for conversion of voice into text.

Further, the conversation capturing device 102 may also implement a suggestion module that may predict and/or suggest a user control command, a context, a text, and may also provide corrections to a predicted context or text, based on current user voice inputs or manual inputs or history of user voice inputs or manual inputs or based on the context. In an exemplary situation, the suggestion module may provide a suggestion beforehand to answer a question, in a web-based online form, based on the context of the question, or based on history of user voice inputs or manual inputs. In an exemplary situation, the suggestion module may provide a suggestion to answer a question, in a web-based online form, based on the context of the question, or based on history of user voice inputs or manual inputs, in cases where the recognition module and the text generator wrongly recognized and predicted the voice input. In another exemplary situation, the suggestion module may correct a wrongly predicted answer by the conversation capturing device 102 or may correct a spelling converted into text, or may provide a better answer to a question, based on the context of the question, or based on current/history of user voice inputs or manual inputs.

The suggestion module may continuously communicate with the word database 108, and may also communicate with the user database 106 for predictions, suggestions and corrections. The word database 108 may also store constrained dictionaries in helping scope the context and text to be recognized by the conversation capturing device 102 based on the possible answers (and their synonyms or alternate terms) that the question is expecting. Thus, the suggestion module may provide situation-based word suggestions and corrections.

In a lot of real time exemplary situations, the recognition module may erroneously recognize the user commands or functions to be performed or the text from the user voice inputs. In such errors by the conversation capturing device 102, often the user tries to re-input the voice commands a number of times, and at last may provide a manual input to perform the function. The reasons for such errors may be numerous, including the failure to recognize the dialect, accent or tone of the user, because the conversation capturing device 102 may have users from diversified language origin. In such error cases, the conversation capturing device 102, including the recognition module, the text generator and the suggestion module, may continuously learn in real time from all the user manual inputs. Thus, the conversation capturing device 102 may provide an online learning system that is developed to enhance the recognition for each user as well as user population (for e.g. Urologists in Kentucky) by learning from instances where user has to make manual corrections on current conversation capturing device's 102 failure to recognize voice commands or answers correctly.

The conversation capturing device 102 may further continuously update the word database 108 (or user specific database) based on the real time learning. Such kind of learning may let the conversation capturing device 102 learn the dialect, pronunciation and accent of the user, every time the user manually provides a corrected command or context or text, thus minimizing recognition errors by the conversation capturing device 102.

The conversation capturing device 102 being a real time learning system from the user's manual inputs may become a highly adaptable system to a wide range of voice accents, pronunciations, tones, modulations in different languages.

Also, the conversation capturing device 102 learns in real time system from the user's manual inputs and eventually learning and adapting the user's voice accents, pronunciations, tones, modulations in his/her language, the conversation capturing device 102 may also provide a personalized user-based voice controlled user interface with the related speech-to-text recognition system. Such personalized user-based user interface would enhance the experience for a regular and a frequent user who regularly uses a software application and/or a device implemented with the present recognition system. Additionally, such personalized user-based user interface would recommend suggestions and corrections to the user based on his/her profile only.

The conversation capturing device 102 may also provide a real-time voice-enabled user interface 104 that is enabled to listen to the user's conversations or voice statements or voice inputs while the conversation capturing device 102 continually trying to comprehend the conversation to automatically predict or suggest future answers or text or context from the conversations using the recognition module in combination with the suggestion module. In an embodiment, the conversation capturing device 102 may implement one or more intent recognition algorithms to automatically answer the questions or predict a user command, or text or context reducing the need for the user to answer them manually.

The conversation capturing device 102 may also include a weightage module that may provide weightage or rankings to the user inputs, such as conversation or answers of the users and also to the users, individually or in combination based on one or more factors including and not limited to a user's profiles, type of user, such as a primary user, a secondary user, a regular and frequent user, trust-based factor, occupation, frequency or importance of user inputs, such as conversation fragments or dialogues related to the user occurring in a conversation, and the like.

In an embodiment, in multi-users conversations, the recognition module may have an ability to identify conversation fragments that belong to each user to be used in picking the most probable and accurate answer recognition through the added trust weightages to users using the weightage module.

The conversation capturing device 102 may allow multiple users to use the voice-enabled user interface 104, listen to real-time multiple users' conversations, comprehend the conversations using the recognition module, provide a trust weightage to the multiple users using the weightage module, in order to determine the most probable and accurate answers/text/context/command based on and from the users' conversations using the suggestion module, thereby further reducing needs for a manual user input.

In an embodiment of the present disclosure, the conversation capturing device 102 may also be implemented in an audio/video listener bot. The listener bot may include user interaction interfaces such as including and not limited to a microphone, a camera and a touch sensitive display screen. The display screen may be touch sensitive or non-touch sensitive. The listener bot may be able to recognize the direct voice inputs from the user, and/or may also be able to recognize and capture automatically user conversations and/or monologues. The users may be a doctor and a patient, where the patient is trying to explain a health-related problem to the doctor. The doctor may use the audio/video listener bot implemented with the conversation capturing device 102 to fill a health-related questionnaire while interacting with and diagnosing problems for each patient, using voice inputs, in order to reduce events of manual inputs. The user (e.g. a doctor) may also be able to provide other user inputs, such as text or image inputs along with the voice inputs into the conversation capturing device 102, using the user input interface 104 such has touch screen display screen or camera. The user may provide such inputs in real-time when the conversation is live.

In such situations, the doctor needs to ask the patient a series of questions to fill in the health-related questionnaire to understand the health-related problem of the patient. In a general scenario, the patient usually explains the problem and further explains his situations or other problems or an event that might have led to the problem, and other such factors and situations. The listener bot implemented with the conversation capturing device 102 automatically listens to the doctor/patient conversation using the voice enabled user interface 104, processes the voice inputs and continuously comprehends the conversations, based on the voice inputs, using the recognition module. In an embodiment, the conversation capturing device 102 may also capture the other types of user inputs in real time using the user input interfaces 104, processes the other types of user inputs, using one or more processing modules or one or more recognition modules, along with processing of the voice inputs, and continuously comprehends the conversations based on the voice inputs in addition to the other types of user inputs. For example, during doctor/patient conversation, the doctor may scribble/write some notes on the touch sensitive screen of the listener bot, along with questioning/talking with the patient. The conversation capturing device 102 may capture this text input from the scribbling of the doctor, process the text input, and comprehend the conversation based on the real-time verbal conversation and real-time text input.

In another example, the doctor may take a picture of a document, using the camera, related to the health-related problem of the patient. The conversation capturing device 102 may capture the image, process the image, identify the context from the image, using the one or more processing modules or one or more recognition modules, and comprehend the conversation based on the real-time verbal conversation and real-time image input.

Thus, the conversation capturing device 102 may capture any type of user input, such as voice, text, image, process the inputs and comprehends them, individually or in combination to predict most probable and accurate answers in the health-related questionnaire.

After or while comprehending the doctor/patient conversation, the conversation capturing device 102 beforehand determines and suggests the most probable and accurate answers to the doctor's series of next questions based on and from the doctor/patient conversation, using the recognition module along with the suggestion module. For e.g. the doctor might have asked a next question related to an earlier event that the patient might have attended, whose answer the doctor needs to fill into the health-related questionnaire using his/her audio/video listener bot. But in the present system 100, the doctor does not need to specifically and manually input the answer or fill the answer to the question specifically using his voice input. The conversation capturing device 102 comprehends the conversations and determines the answer to the next question, and automatically either fills or suggests to fill in the health-related questionnaire. Thus, saving energy, skill and time of the doctor.

Also, while comprehending the conversation between the doctor and the patient, the conversation capturing device 102 may capture inputs from the doctor and the patient, such as the verbal conversation, to identify fragments of the conversation related to each user, e.g. conversation fragments spoken by the doctor and by the patient. The conversation capturing device 102 may provide weightage to the user inputs, e.g. the conversation fragments of the doctor and the patient, in real-time, to identify the most accurate and probable answer to a question. In addition, the conversation capturing device 102 may also already have a weightage or ranking provided to the user, such as a doctor, which it may use for identifying the most accurate and probable answer to a question.

In some embodiments, the conversation capturing device 102 may further include a processor, which may be communicatively coupled to a memory. The memory may store processor instructions, which when executed by the processor may cause the processor to capture conversation between users. This is further explained, in detail in conjunction with FIGS. 2-12. The processor in conjunction with the memory may perform various functions including receiving voice inputs, segregating the voice input, identifying context, generating text, classification of voice fragments, identity determination, and the like.

The memory may store various data that may be captured, processed, and/or required by the conversation capturing device 102. The memory may be a non-volatile memory or a volatile memory. Examples of non-volatile memory, may include, but are not limited to a flash memory, a Read Only Memory (ROM), a Programmable ROM (PROM), Erasable PROM (EPROM), and Electrically EPROM (EEPROM) memory. Examples of volatile memory may include, but are not limited to, Dynamic Random-Access Memory (DRAM), and Static Random-Access memory (SRAM).

It may be understood that the above example is only one exemplary situation in which the present conversation capturing device 102 may be applied, and this should not restrict the applications of the conversation capturing device 102.

Referring to FIG. 2, a block diagram of the exemplary conversation capturing device 102 is illustrated, in accordance with some embodiments of the present disclosure. The conversation capturing device 102 may be configured to capture conversation between a plurality of users. The conversation capturing device 102 may include a processor and a memory. Further, the memory of the conversation capturing device 102 may include various module including, but not limited to, a segregation module 204, a conversion module 206, a context identification module 208, a classification module 210, a mapping module 212 an identity determination module 214, and a profile updating module 216. The conversion module 206 is referred to as the text generation module in some embodiments of the present invention, Further, each of the module 204-216 may include various sub-modules, such as the classification module 210 may include the weightage module. The memory may further include a database 218 to store the information and intermediate results generated by the conversation capturing device 102. The conversation capturing device 102 may also include a recognition module as explained in FIG. 1.

The segregation module 204 may be configured to receive voice inputs 202 from a first user and a second user. Here, the first user and the second user may be a combination of at least one of a patient-doctor combination, a litigant-lawyer combination, a buyer-seller combination, a teacher-student combination, and so on. The combinations of users may depend upon the application of the conversation capturing device 102. The system 200 may include one or more microphones for capturing the voice inputs 202 of users. The microphones may transmit the voice inputs 202 to the segregation module 204. The microphones are positioned in the vicinity of each of the first user and the second user. Further, the voice inputs 202 may include voice attributes. And, the voice attributes may include at least one of an accent of speech, a degree of loudness of speech, a speed of speech, and a tone of speech, and wherein the method further comprises detecting the voice attributes.

Further, the segregation module 204 may be further configured to segregate the voice inputs 202 into a plurality of voice-fragments. It should be noted that each of the plurality of voice-fragments is associated with one of the first user and the second user. The segregation module 204 may be communicatively coupled to the conversion module 206.

The conversion module 206 may convert the plurality of voice-fragments into a plurality of text inputs. The conversion module 206 may include a voice-to-text conversion model (not shown in FIG. 2) for text conversion. The conversion module 206 may be operatively coupled to the context identification module 208. The context identification module 210 may receive the input from conversion module 206 and subsequently identify the context of the conversation from the plurality of text inputs. Further, the context identification module 210 may be communicatively connected to the classification module 212.

The classification module 212 may be configured to classify the voice-fragments into a first-user category and a second user category based on the voice attributes and the context of the conversation. It should be noted that the first-user category is associated with the voice-fragments received from the first user and the second-user category is associated with the voice-fragments received from the second user.

In some embodiments, the classification module 212 may identify one or more keywords from the plurality of text inputs corresponding to the plurality of voice-fragment. Further, the classification module 212 may assign a weightage to each of the plurality of voice-fragments based on the one or more keywords. Further, the classification module 212 may classify the voice-fragments into the first-user category and the second user category based on the voice attributes and the weightage assigned to each of the plurality of voice-fragments.

For example, the classification module 212 may classify the more frequently speaker as doctor, i.e. if frequency of voice attributes detected over a period of time is greater than a threshold frequency, then the corresponding user is classified as second category (doctor). As illustrated in FIG. 2, the classification module 212 may be further communicatively coupled to the mapping module 212 and the database 218.

The mapping module 212, in some embodiments, may be configured to fetch a plurality of user profiles from a database. It should be noted that each of the plurality of user profiles may include at least one of: a user data and a voice sample of the user. Also, the mapping module 212 may be configured to map the context of the conversation with user data of the plurality of user profiles, the first-user category voice-fragments with the voice samples of the plurality of user profiles. The mapping module 212 may transmit the mapped data to the coupled identity determination module 214

The identity determination module 214 may be configured for determining an identity of the user based on the mapping. The identity determination module 214 may be coupled to the profile updating module 216. The profile updating module 214 may exchange (i.e., receive or transmit) the information with the context identification module 208, identity determination module 214, and the database 218. Further, the profile updating module 216 may be configured for updating the user profile of the identified user based on the context of the conversation. It should be noted that the user profiles may include one or more predefined fields. The one or more predefined fields may include a name of the user, a residing location of the user, a birth location of the user, an occupation of the user, a language known to the user, a preferred language of the user, a dialect spoken by the user, a past issue of the user, and a present issue of the user.

In some embodiments the profile updating module 216 may populate the one or more predefined fields in the user profile of the identified user using the text inputs corresponding to first user-category voice-fragments, to update the user profile.

In some embodiments, the profile updating module 216, during a conversation, may receive secondary inputs from the one or more users. The secondary inputs include at least one of aa text input or an image including a handwritten text. Further, the profile updating module 216 may populate the one or more predefined fields of the user profile of the identified user based on the secondary inputs.

The conversation capturing device 102 may also include navigation module (not shown in FIG. 2) for identifying a navigating command from the second user-category voice fragments, and navigating from a first graphical user interface (GUI) component of an application to a second GUI. Also, the conversation capturing device 102 may include a prediction model (not shown in FIG. 2) for predicting a text input for the second user based on the context of the conversation and the word database 108, upon receiving a voice input from a first user and classifying the voice-fragments of the voice input into the first-user category and the second user category.

Additionally, the conversation capturing device may include a suggestion module (not shown in FIG. 2) which displays a suggestion for populating the one or more predefined fields using the predicted text input for the second user. In some, a validation for the suggestion may be received from the first user using the suggestion module.

In some embodiments, the one or more predefined fields may be populated using the predicted text input for the second user.

The conversation capturing device may also include a correction module (not shown in FIG. 2). The correction module may receive a corrective input from the first user upon providing the suggestion. The corrective input includes one of a text input or a voice input. In some embodiments, the one or more predefined fields may be populated based on the corrective input overriding the suggestion, and the word database 108 may be updated with the corrective input, using the correction module.

Further, it should be noted that, the conversation capturing device 102 may be implemented in programmable hardware devices such as programmable gate arrays, programmable array logic, programmable logic devices, or the like. Alternatively, the system 100 and associated conversation capturing device 102 may be implemented in software for execution by various types of processors. An identified engine/module of executable code may, for instance, include one or more physical or logical blocks of computer instructions which may, for instance, be organized as a component, module, procedure, function, or other construct. Nevertheless, the executables of an identified engine/module need not be physically located together but may include disparate instructions stored in different locations which, when joined logically together, comprise the identified engine/module and achieve the stated purpose of the identified engine/module. Indeed, an engine or a module of executable code may be a single instruction, or many instructions, and may even be distributed over several different code segments, among different applications, and across several memory devices.

As will be appreciated by one skilled in the art, a variety of processes may be employed for providing security and access control. For example, the exemplary system 100 and associated conversation capturing device 102 may create the process plan, by the process discussed herein. In particular, as will be appreciated by those of ordinary skill in the art, control logic and/or automated routines for performing the techniques and steps described herein implemented by the system 100 and the associated conversation capturing device 102 either by hardware, software, or combinations of hardware and software. For example, suitable code may be accessed and executed by the one or more processors on the system 100 to perform some or all of the techniques described herein. Similarly, application specific integrated circuits (ASICs) configured to perform some or all the processes described herein may be included in the one or more processors on the system 100.

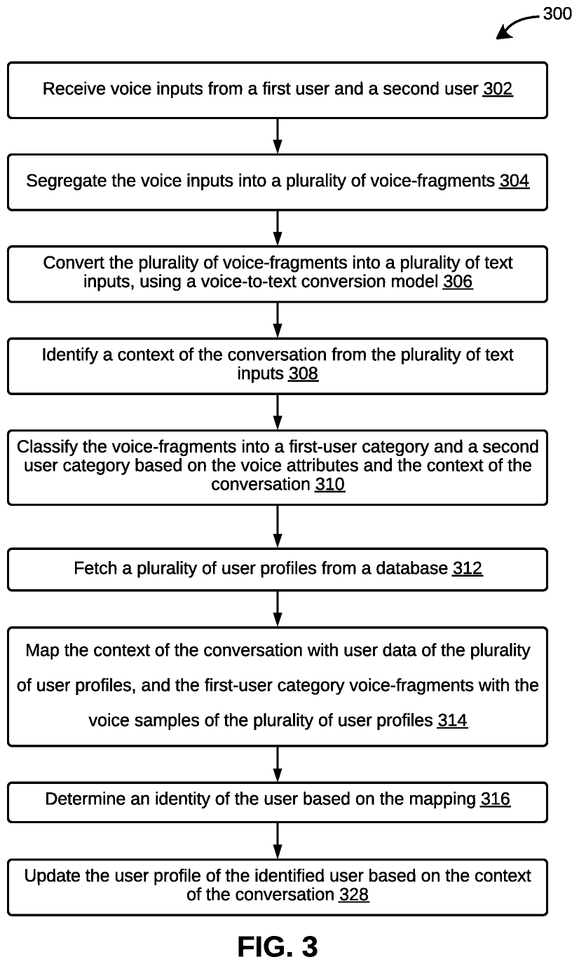

Referring now to FIG. 3, a method 300 for capturing a conversation between a plurality of users is depicted via a flow diagram, in accordance with some embodiments of the present disclosure. Steps of the method 300 are performed using various modules of the conversation capturing device 102. FIG. 3 is explained in conjunction with FIGS. 1-2.

At step 302, voice inputs may be received. The voice inputs may be received from users, i.e., a first user and a second user. For example, in some embodiments the first user and the second user may be a patient and a doctor. In some other embodiments, the first user and the second user may be a litigant and a lawyer. Additionally, in some embodiments the first and the second user may be a buyer and a seller. In an embodiment, the first user and the second user may also be other combinations of the users, such as a teacher and a student. It should be noted that the voice inputs may be received by the conversation capturing device 102 via one or more microphones. In other words, the one or more microphones may capture the voice of the users (i.e., voice of the users within vicinity of the microphone) when they speak, and process the voice as an input to the conversation capturing device 102. Also, it should be noted that the voice inputs include voice attributes.

The voice attributes may include, but not limited to, an accent of speech, a degree of loudness of speech, a speed of speech, and a tone of speech, and wherein the method further comprises detecting the voice attributes.

At step 304, the voice inputs may be segregated into a plurality of voice-fragments. It should be noted that the segregation module 204 may be responsible for executing the step 304. Here, each of the plurality of voice-fragments may be associated with one of the first user and the second user. At step 306, the plurality of voice-fragments may be converted into a plurality of text inputs, using a voice-to-text conversion model of the conversion module 206.

At step 308, a context of the conversation may be identified from the plurality of text inputs using the context identification module 208. Thereafter, at step 310, the voice-fragments may be classified into a first-user category and a second user category. The classification may be performed based on the voice attributes and the context of the conversation. The first-user category may be associated with the voice-fragments received from the first user. The second-user category may be associated with the voice-fragments received from the second user. It should be noted that classification may be performed using the classification module 210.

At step 312, a plurality of user profiles may be fetched from a database (same as the database 218). It should be noted that each of the plurality of user profiles may include at least one of a user data and a voice sample of the user. The plurality of user profiles may include one or more predefined fields. The context of the conversation may be mapped with user data of the plurality of user profiles, and the first-user category voice-fragments may be mapped with the voice samples of the plurality of user profiles, at step 314. Mapping may be performed by the mapping module 212.

At step 316, an identity of the user may be determined using the identity determination module 214. It may be noted that, mapping may be considered to determine the identity. At step 318, the user profile of the identified user may be updated based on the context of the conversation. The profile updating module 216 may be used to perform this step. In some embodiments, the one or more predefined fields may be populated in the user profile of the identified user using the text inputs corresponding to first user-category voice-fragments, to update the user profile. The one or more predefined fields may include a name of the user, a residing location of the user, a birth location of the user, an occupation of the user, a language known to the user, a preferred language of the user, a dialect spoken by the user, a past issue of the user, and a present issue of the user.

In some embodiments, a navigating command from the second user-category voice fragments may be identified. Further, upon identification of navigating command, navigation from a first graphical user interface (GUI) component of an application to a second GUI may be performed.

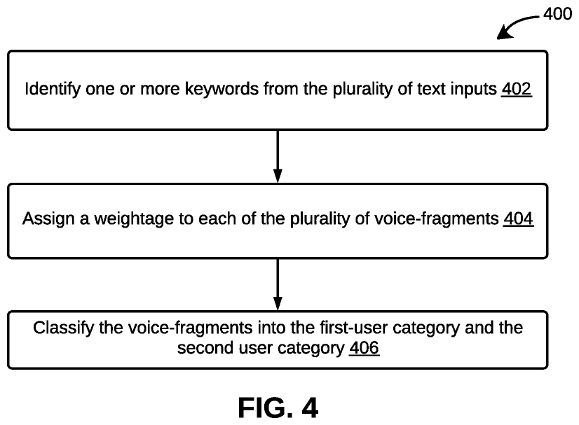

Referring now to FIG. 4, a method 400 for classifying the voice-fragments into different categories is depicted via a flow diagram, in accordance with some embodiments of the present disclosure. Each step of the method 400 may be performed by the classification module 210 of the conversation capturing device 102. FIG. 4 is explained in conjunction with FIGS. 1-3.

At step 402, one or more keywords may be identified from the plurality of text inputs corresponding to the plurality of voice-fragments. At step 404, a weightage may be assigned to each of the plurality of voice-fragments. It should be noted that one or more keywords may be considered to assign the weightage. It should be noted that the weightage module as explained in previous Figures. may be responsible to perform this step. At step 406, the voice-fragments may be classified into the first-user category and the second user category. The classification may performed based on the voice attributes and the weightage assigned to each of the plurality of voice-fragments. For example, classifying a doctor as a more frequent speaker, i.e. if frequency of the voice attributes detected over a period of time is greater than a threshold frequency, then a corresponding user may be classified as second category (doctor).

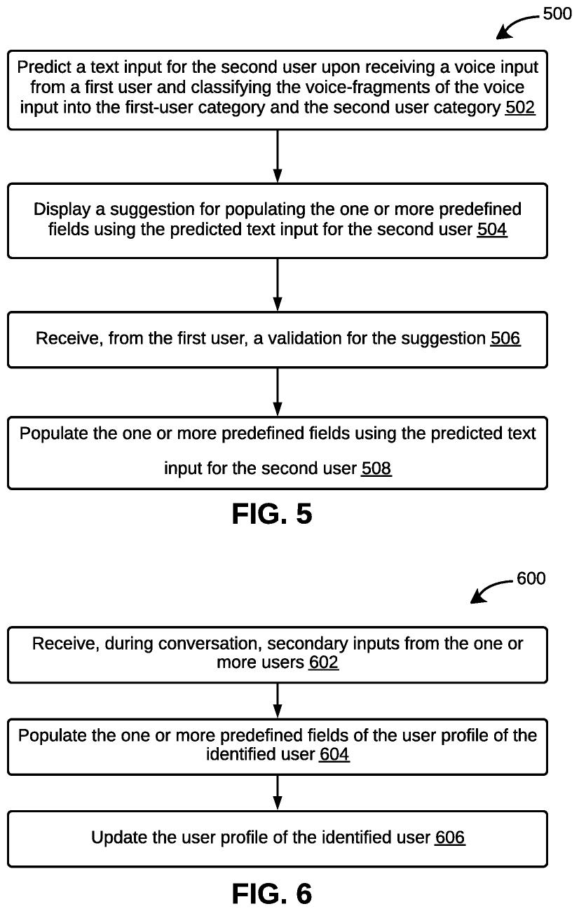

Referring now to FIG. 5, a method 500 for populating the one or more predefined fields is depicted via a flow diagram, in accordance with some embodiments of the present disclosure. FIG. 5 is explained in conjunction with FIGS. 1-4.

At step 502, a text input for the second user may be predicted by a prediction module, upon receiving a voice input from a first user and classifying the voice-fragments of the voice input into the first-user category and the second user category. It should be noted that prediction may be performed based on based on the context of the conversation and a word database (same as the word database 108). Thereafter, at step 504, a suggestion for populating the one or more predefined fields may be displayed on a user interface based on the predicted text input for the second user. Further, a validation for the suggestion may be received from the first user, at step 506. At step 508, the one or more predefined fields may be populated based on the predicted text input for the second user.

In an alternate embodiment, a corrective input from the first user may be received upon providing the suggestion. The corrective input may include one of a text input or a voice input. Further, based on the corrective input the one or more predefined fields may be populated overriding the suggestion. Thereafter, the word database 108 may be updated with the corrective input.

Referring now to FIG.6, a method 600 for updating the user profile of the identified user is depicted via a flow diagram, in accordance with some embodiments of the present disclosure. Each step of the method 600 may be performed using profile the updating module 216. FIG. 6 is explained in conjunction with FIGS. 1-5. At step 602, secondary inputs may be received from the one or more users during the conversation. It should be noted that the secondary input may be at least one of a text input or an image. Further, the image may include a handwritten text. After that, the one or more predefined fields of the user profile of the identified user may be populated based on the secondary inputs.

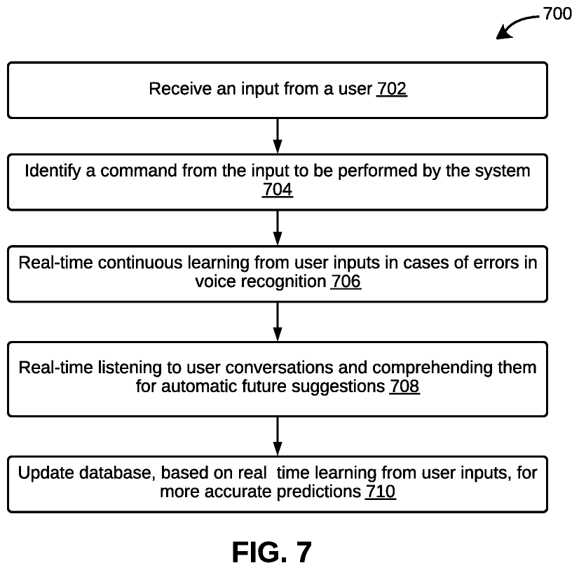

Referring now to FIG. 7, a method 700 implementing a system of capturing conversation is depicted via a flow diagram, in accordance with some embodiments of the present disclosure. Each step of the method 700 may be performed by the conversation capturing device 102. FIG. 7 is explained in conjunction with FIGS. 1-6.

Steps shown in the method 700 in the FIG. 7 may or may not follow a flow as shown in the flowchart in the FIG. 7. Thus, the flow of steps in the method 700 should not be restricted as shown in the FIG. 7.

The method 700 may comprise a step 702 of receiving, by the user interface 104, an input from a user. Further, at a step 704, using the recognition module, a user command may be identified from the user input to perform one or more functions by the conversation capturing device 102. The user commands and the related one or more functions that may be recognized and performed by the the conversation capturing device 102 102 may include and is not limited to recognizing a user control command from the user input to control a function attribute of a device or an application which implements the conversation capturing device 102; recognizing a selection command from the user input to select an attribute in a device or an application which implements the conversation capturing device 102; recognizing a text command from a voice input to identify a voice input to be converted into text; recognizing a navigating command from the user input to navigate over one graphical UI of an application or between multiple GUIs of an application. It may be apparent to a person skilled in the art that the conversation capturing device 102 may perform more than one functions, apart from the aforementioned, based on the captured user inputs and processing of the user inputs, without deviating from the meaning and scope of the present disclosure.

Further, at a step of 706, the conversation capturing device 102 may execute one or more modules, such as the recognition module, the text generator, the suggestion module, either individually or in combination for real-time continuous learning from the user inputs in cases of prediction errors by the conversation capturing device 102, as explained in details in the FIG. 1.

Additionally, at a step of 708, the conversation capturing device 102 may execute one or more modules, such as the recognition module, the text generator, the suggestion module, either individually or in combination for real-time capturing or listening, using the user interface 104, to user inputs, such as user conversations and comprehending them, using one or more modules, such as the recognition module, the text generator, the suggestion module, either individually or in combination, for automatic future suggestions and corrections, as explained in details in the FIG. 1.

At a step of 710, the conversation capturing device 102 may update word databases 108, based on real time learning from user inputs, for more accurate future corrections and suggestions.

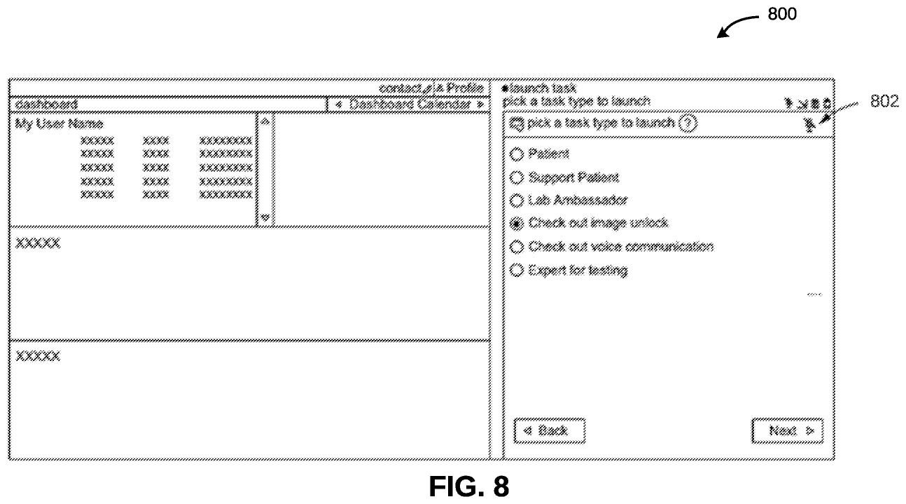

Referring now to FIGS. 8-12, exemplary graphical user interfaces [GUIs] with a voice enabled user interface, implementing the conversation capturing device 102, showing an example of a user interacting with the graphical user interfaces through voice input are illustrated, in accordance with some embodiments of the present disclosure. The description of the FIGS. 8-12 should be read and understood in conjunction with the conversation capturing device 102 and methods 300-700 as explained in the FIGS. 1-7 above, in accordance with an embodiment of the disclosure. The FIGS. 8-12 may include at least one limitation of the conversation capturing device 102 as explained in the FIG. 1 above, in accordance with an embodiment of the disclosure.

FIGS. 8-12 shows exemplary GUIs that represents a health-related questionnaire, in an exemplary doctor/patient situation, where the doctor needs to fill in the questionnaire for diagnosing a problem in a patient. The doctor may himself fill health related questionnaire using his voice inputs, or as explained earlier, the conversation capturing device 102 may comprehend the doctor/patient conversation and auto-fill or auto-suggest to fill in the questionnaire.

The exemplary GUI 800, as shown in the FIG. 8, may include a voice enabled user interface 802 (that may function similarly to a voice enabled user interface 104) to capture user voice inputs. As illustrated in FIG. 8, the GUI 800 may include “pick a task type launch” tab, which may further include various options, such as “a patient”, “support patient”, “lab ambassador”, “check out image unlock”, “check out voice communication”, and “expert for testing”. For an example, a doctor may provide a voice input against a tab “pick a task type to launch”, by saying “intake patient”. The voice enabled user interface 802 captures the voice input “intake patient”, and the recognition module selects “intake patient” at the tab “pick a task type to launch” over the GUI 800. Similarly, as illustrated in FIG. 8, when a user provides voice input by saying check out image unlock, then the recognition module of the conversation capturing device may select “Check out image unlock” provided at the tab “pick a task type to launch” over the GUI 800.

In an embodiment, the doctor may provide a voice input “next page” at this stage to command the recognition module to navigate the questionnaire to the next page. In another embodiment, as soon as the recognition module selects “intake patient”, the recognition module automatically navigates the questionnaire to the next page.

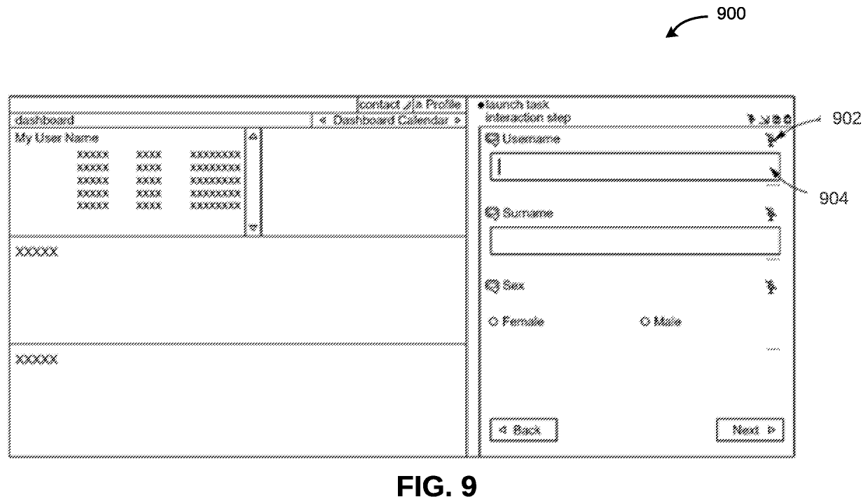

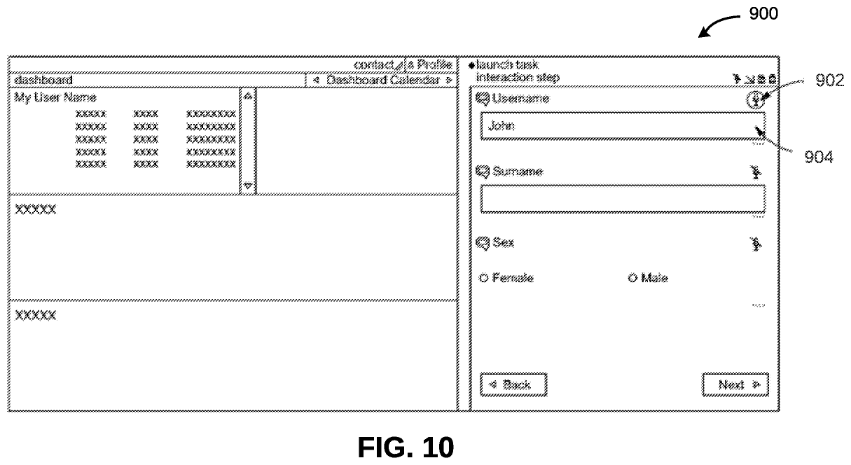

The exemplary GUI 900, as shown in the FIGS. 9-10, may include a voice enabled user interface 902 (that may function similarly to voice enabled user interface 104) to capture user voice inputs. Also, as illustrated at the GUI 900, each tab or question has a voice enabled user interface to answer that particular question. While answering a particular question, the voice enabled user interface associated to that question gets automatically activated, while the other voice enabled user interfaces becomes inactive [as shown in FIG. 10]. This way, the conversation capturing device 102 and/or the user are able to identify which particular question is being answered.

To answer a tab 904 at the GUI 900, the voice enabled user interface 902 associated to the tab 904 gets activated. The doctor may provide a voice input against the tab “first name”, by saying patient's name e.g. “John”. The voice enabled user interface 902 captures the voice input “John”, and the recognition module with the text generator fills in the tab 904 with the name “John” over the GUI 900. Similarly, each tab at the GUI 900 (also the similar other GUIs with such forms) may be filled or selected.

In an embodiment, the doctor may provide a voice input “next question” at this stage to command the recognition module to navigate the questionnaire to the next question. The conversation capturing device 102 activates the voice enabled interface associated to the next question. In an embodiment, the doctor may provide a voice input “next page” at this stage to command the recognition module to navigate the questionnaire to the next page. In another embodiment, as soon as the recognition module fills/selects in the last tab of such questionnaire, the recognition module automatically navigates the questionnaire to the next page.

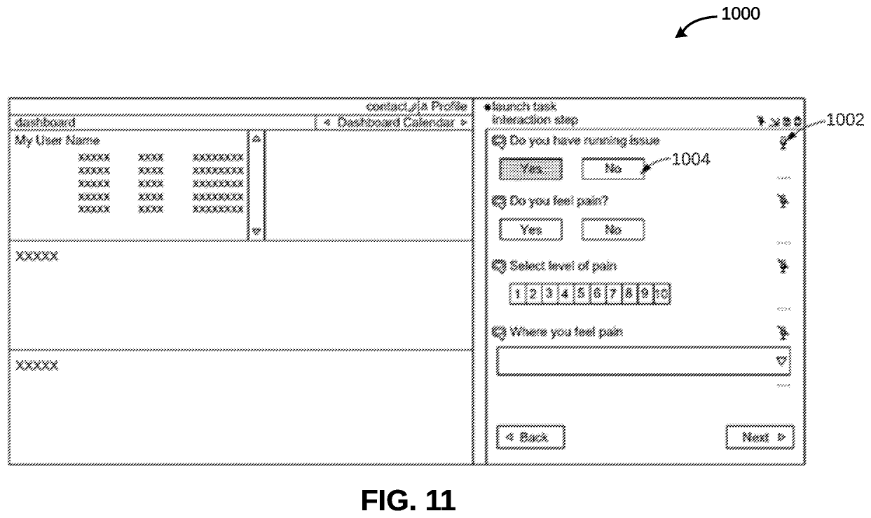

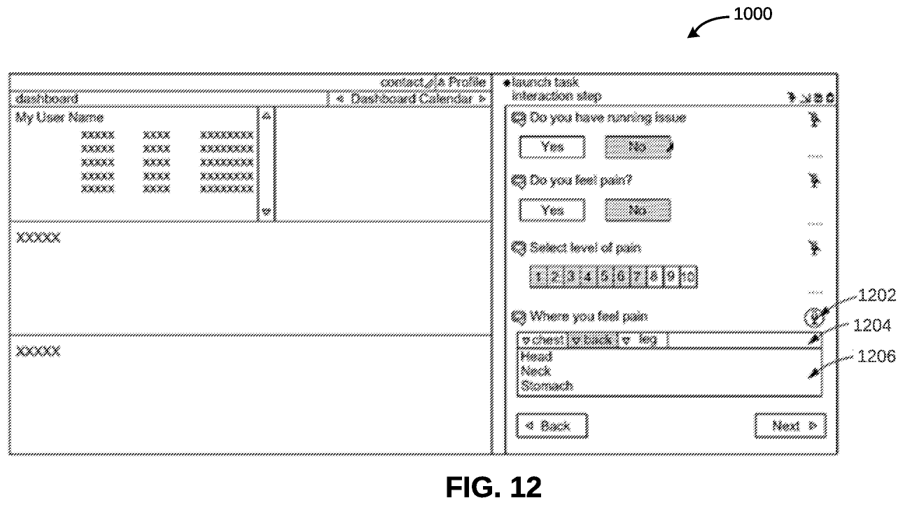

The exemplary GUI 1000, as shown in the FIGS. 11-12, may include a voice enabled user interface 1002 (that may function similarly to voice enabled user interface 104) to capture user voice inputs. To answer a tab 1004 at the GUI 1000, the voice enabled user interface 1002 associated to the tab 1004 gets activated. The doctor may provide a voice input against the tab/question 1004 “do you have running nose?”, by saying “yes”. The voice enabled user interface 1002 captures the voice input “yes”, and the recognition module selects “yes” against the question 1004 over the GUI 1000. Similarly, each tab at the GUI 1000 (also the similar other GUIs with such forms) may be filled or selected.

In an embodiment, as soon as the doctor provides a voice input “next question”, the conversation capturing device 102 navigates the questionnaire to the next question. Thus, till the doctor says “next question”, the doctor has an opportunity to change the answer multiple times before finalizing. In an embodiment, as soon as the doctor provides an answer to a question, the conversation capturing device 102 navigates the questionnaire to the next question.

In the FIG. 12, to answer the tab or question 1204, the voice enabled user interface 1202 associated to it get activated. At this stage, the voice enabled user interface 1202 captures the voice input e.g. “chest”, “leg”, “foot” etc., and the recognition module with the text generator fills in the tab 1204 with the answer “chest”, “leg”, “foot” over the GUI 1200. Additionally, the voice enabled user interface 1202, the recognition module with the suggestion module may also listen to doctor/patient conversation, and may comprehend the answer to the question 1204, and may suggest an answer, or may auto-fill in the answer. Also, while suggesting answers, the suggestion module may scope the context and text to provide the possible answers (and their synonyms or alternate terms) that the question is expecting.

Additionally, there might be case while filling in the questionnaire, the recognition module is unable to recognize the voice input and unable to fill in the answer for a question. At this stage, the doctor may try a number of times of providing the same voice input, but at a failed attempt to be recognized by the recognition module, the doctor may provide a manual input, for e.g. manually selecting an answer from the drop-down menu 1206 at the question 1204.

The conversation capturing device 102 now uses its real time learning to learn from this instance to correctly recognize in the future, and also to provide correct suggestions in the future. Also, the conversation capturing device 102 at this stage automatically learns in real time about the dialect, pronunciation and accent of the user from the corrected manual user input against the incorrect voice recognition. In an exemplary situation, the doctor may provide various voice inputs to select/fill in the questionnaire or navigate through the questionnaire, such as “next question”, “previous question”, “skip question”, “next page”, “previous page”, “skip page”, “suggest answer”, “auto-fill tab”, “repeat”, “delete”, etc., while controlling, navigating, answering the questionnaire represented over the GUIs. It may be apparent to a person skilled in the art that there may be different types of voice command that a user may input for controlling and/or navigating and/or answering and/or providing speech-text inputs through the graphical user interfaces, without deviating from the meaning and scope of the present disclosure.

Thus, a doctor is able to fill the health-related questionnaire using voice inputs. It may be understood that the GUIs shown in the FIGS. 8-12 are only to provide examples for understanding, and under no circumstance, the conversation capturing device 102 should be restricted to the GUIs shown in the FIGS. 8-12.

Advantageously, the present disclosure provides a faster and hands free way to capture information increasing the productivity of the users while also reducing the physical fatigue and possible injury from operating keyboard and mouse. The present disclosure provides an ability to automatically infer the answers to questions from the conversation text and tonality of the voice may not only increase the productivity of the users further, but, also let the answers be captured as they come (possibly, out of order), e.g., in natural conversations.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although the present invention has been described in connection with some embodiments, it is not intended to be limited to the specific form set forth herein. Rather, the scope of the present invention is limited only by the claims. Additionally, although a feature may appear to be described in connection with particular embodiments, one skilled in the art would recognize that various features of the described embodiments may be combined in accordance with the invention.

Furthermore, although individually listed, a plurality of means, elements or process steps may be implemented by, for example, a single unit or processor. Additionally, although individual features may be included in different claims, these may possibly be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate.

What is claimed is:

1. A method of capturing a conversation between a plurality of users, the method comprising:
receiving voice inputs from a first user and a second user, wherein the voice inputs are obtained using one or more microphones positioned in the vicinity of each of the first user and the second user, wherein the voice inputs comprise voice attributes;
segregating the voice inputs into a plurality of voice-fragments, wherein each of the plurality of voice-fragments is associated with one of the first user and the second user;
converting the plurality of voice-fragments into a plurality of text inputs, using a voice-to-text conversion model;
identifying a context of the conversation from the plurality of text inputs;
classifying the voice-fragments into a first-user category and a second user category based on the voice attributes and the context of the conversation, wherein the first-user category is associated with the voice-fragments received from the first user and the second-user category is associated with the voice-fragments received from the second user;
fetching a plurality of user profiles from a database, wherein each of the plurality of user profiles comprises at least one of: a user data and a voice sample of the user;
mapping:
the context of the conversation with user data of the plurality of user profiles; and
the first-user category voice-fragments with the voice samples of the plurality of user profiles;
determining an identity of the user based on the mapping; and
updating the user profile of the identified user based on the context of the conversation.

2. The method of claim 1, wherein the voice attributes comprises at least one of an accent of speech, a degree of loudness of speech, a speed of speech, and a tone of speech, and wherein the method further comprises detecting the voice attributes.

3. The method of claim 1, wherein each of the plurality of user profiles comprises one or more predefined fields, and wherein updating the user profile of the identified user comprises populating the one or more predefined fields in the user profile of the identified user using the text inputs corresponding to first user-category voice-fragments, to update the user profile.

4. The method of claim 1, further comprising:
identifying a navigating command from the second user-category voice fragments; and
navigating from a first graphical user interface (GUI) component of an application to a second GUI.

5. The method of claim 1, wherein the one or more predefined fields comprise:
a name of the user, a residing location of the user, a birth location of the user, an occupation of the user, a language known to the user, a preferred language of the user, a dialect spoken by the user, a past issue of the user, and a present issue of the user.

6. The method of claim 1, wherein classifying the voice-fragments into the first-user category and the second user category comprises:
identifying one or more keywords from the plurality of text inputs corresponding to the plurality of voice-fragments;
assigning a weightage to each of the plurality of voice-fragments based on the one or more keywords; and
classifying the voice-fragments into the first-user category and the second user category based on the voice attributes and the weightage assigned to each of the plurality of voice-fragments.

7. The method of claim 1, further comprising:
upon receiving a voice input from a first user and classifying the voice-fragments of the voice input into the first-user category and the second user category, predicting a text input for the second user based on the context of the conversation and a word database;
displaying, on a user interface, a suggestion for populating the one or more predefined fields using the predicted text input for the second user;
receiving, from the first user, a validation for the suggestion; and
populating the one or more predefined fields using the predicted text input for the second user.

8. The method of claim 7, further comprising:
upon providing the suggestion, receiving a corrective input from the first user, wherein the corrective input comprises one of a text input or a voice input;
populating the one or more predefined fields based on the corrective input overriding the suggestion; and
updating the word database with the corrective input.

9. The method of claim 1, wherein updating the user profile of the identified user comprises:
receiving, during conversation, secondary inputs from the one or more users, wherein the secondary inputs comprise one of:
a text input; or
an image comprising a handwritten text; and
populating the one or more predefined fields of the user profile of the identified user based on the secondary inputs.

10. A system for capturing a conversation between a plurality of users, the system comprising:
a processor; and
a memory communicatively coupled to the processor, wherein the memory stores processor-executable instructions, which, on execution, cause the processor to:
receive voice inputs from a first user and a second user, wherein the voice inputs are obtained using one or more microphones positioned in the vicinity of each of the first user and the second user, wherein the voice inputs comprise voice attributes;
segregate the voice inputs into a plurality of voice-fragments, wherein each of the plurality of voice-fragments is associated with one of the first user and the second user;
convert the plurality of voice-fragments into a plurality of text inputs, using a voice-to-text conversion model;
identify a context of the conversation from the plurality of text inputs;
classify the voice-fragments into a first-user category and a second user category based on the voice attributes and the context of the conversation, wherein the first-user category is associated with the voice-fragments received from the first user and the second-user category is associated with the voice-fragments received from the second user;
fetch a plurality of user profiles from a database, wherein each of the plurality of user profiles comprises at least one of: a user data and a voice sample of the user;
map:
the context of the conversation with user data of the plurality of user profiles; and
the first-user category voice-fragments with the voice samples of the plurality of user profiles;
determine an identity of the user based on the mapping; and
update the user profile of the identified user based on the context of the conversation.

11. The system of claim 10, wherein the voice attributes comprises at least one of an accent of speech, a degree of loudness of speech, a speed of speech, and a tone of speech, and wherein the method further comprises detecting the voice attributes.

12. The system of claim 10, wherein each of the plurality of user profiles comprises one or more predefined fields, and wherein updating the user profile of the identified user comprises populating the one or more predefined fields in the user profile of the identified user using the text inputs corresponding to first user-category voice-fragments, to update the user profile.

13. The system of claim 10, wherein the processor-executable instructions further cause the processor to:
identify a navigating command from the second user-category voice fragments; and
navigate from a first graphical user interface (GUI) component of an application to a second GUI.

14. The system of claim 10, wherein the one or more predefined fields comprise a name of the user, a residing location of the user, a birth location of the user, an occupation of the user, a language known to the user, a preferred language of the user, a dialect spoken by the user, a past issue of the user, and a present issue of the user.

15. The system of claim 10, wherein the processor-executable instructions further cause the processor to classify the voice-fragments into the first-user category and the second user category by:
identifying one or more keywords from the plurality of text inputs corresponding to the plurality of voice-fragments;
assigning a weightage to each of the plurality of voice-fragments based on the one or more keywords; and
classifying the voice-fragments into the first-user category and the second user category based on the voice attributes and the weightage assigned to each of the plurality of voice-fragments.

16. The system of claim 10, wherein the processor-executable instructions further cause the processor to:
upon receiving a voice input from a first user and classifying the voice-fragments of the voice input into the first-user category and the second user category, predict a text input for the second user based on the context of the conversation and a word database;
display, on a user interface, a suggestion for populating the one or more predefined fields using the predicted text input for the second user;
receive, from the first user, a validation for the suggestion; and
populate the one or more predefined fields using the predicted text input for the second user.

17. The system of claim 16, wherein the processor-executable instructions further cause the processor to:
upon providing the suggestion, receive a corrective input from the first user, wherein the corrective input comprises one of a text input or a voice input;
populate the one or more predefined fields based on the corrective input overriding the suggestion; and
update the word database with the corrective input.

18. The system of claim 10, wherein the processor-executable instructions further cause the processor to wherein update the user profile of the identified user by:
receiving, during conversation, secondary inputs from the one or more users, wherein the secondary inputs comprise one of:
a text input; or
an image comprising handwritten text; and
populating the one or more predefined fields of the user profile of the identified user based on the secondary inputs.

19. A non-transitory computer-readable medium storing computer-executable instructions for capturing a conversation between a plurality of users, the computer-executable instructions configured for:
receiving voice inputs from a first user and a second user, wherein the voice inputs are obtained using one or more microphones positioned in the vicinity of each of the first user and the second user, wherein the voice inputs comprise voice attributes;
segregating the voice inputs into a plurality of voice-fragments, wherein each of the plurality of voice-fragments is associated with one of the first user and the second user;
converting the plurality of voice-fragments into a plurality of text inputs, using a voice-to-text conversion model;
identifying a context of the conversation from the plurality of text inputs;
classifying the voice-fragments into a first-user category and a second user category based on the voice attributes and the context of the conversation, wherein the first-user category is associated with the voice-fragments received from the first user and the second-user category is associated with the voice-fragments received from the second user;
fetching a plurality of user profiles from a database, wherein each of the plurality of user profiles comprises at least one of: a user data and a voice sample of the user;
mapping:
the context of the conversation with user data of the plurality of user profiles; and
the first-user category voice-fragments with the voice samples of the plurality of user profiles;
determining an identity of the user based on the mapping; and
updating the user profile of the identified user based on the context of the conversation.

20. The non-transitory computer-readable medium of the claim 19, wherein the computer-executable instructions further configured for:
identifying a navigating command from the second user-category voice fragments; and
navigating from a first graphical user interface (GUI) component of an application to a second GUI.
